(19) United States
(12) Patent Application Publication
(10) Pub. No.: US 2004/0266699 A1
(43) Pub. Date: Dec. 30, 2004

(54) Flavonoid compounds capable of modifying the dynamic and/or physical state of biological membranes and to stimulate the endogenous synthesis of stress proteins in eukaryotic cells, relative synthesis and their use

(76) Inventor: Amalia Porta, Crotone (IT)

Correspondence Address: WILLIAM COLLARD, COLLARD & ROE, P.C., 1077 NORTHERN BOULEVARD, ROSLYN, NY, 11576, US

(21) Appl. No.: 10/491,612

(22) PCT Filed: Oct. 4, 2002
(86) PCT No.: PCT/EP02/11181

(30) Foreign Application Priority Data
Oct. 4, 2001 (IT) RM2001A000600

(51) Int. Cl. A61K 31/7048; A61K 31/353; C07H 15/00

(52) U.S. Cl. 514/27; 536/8; 514/456

(57) ABSTRACT

The invention relates to flavonoids compounds of formula (I) and (II) capable of modifying the dynamic and/or physical state of biological membranes and to stimulate the endogenous synthesis of stress proteins in eukaryotic cells. Such compounds are molecules of plant origin or synthetic. The invention also describes a method to identify, purify and chemically synthesize such flavonoid compounds and test their efficacy through their capacity to stimulate the transcription of stress genes and as a consequence, to interact with biological membranes with alteration of their relative physical state. Such compounds and corresponding pharmaceutically acceptable derivatives and/or salts have applications in the areas of pharmaceuticals, more specifically in cosmetics and dermatology, for all those afections related to an alteration of the expression of stress genes.

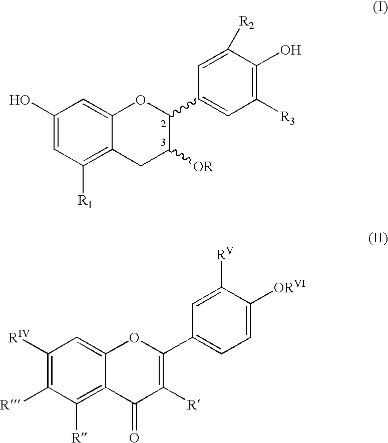

### FIELD OF THE INVENTION

The present invention relates to flavonoid compounds capable of modifying the dynamic and/or physical state of biological membranes and to stimulate the endogenous synthesis of stress proteins in eukaryotic cells, relative synthesis and their use. In particular, such compounds are molecules of plant origin or synthetic. The invention also describes a method to identify, purify and chemically synthesize such flavonoid compounds and test their efficacy through their capacity to stimulate the transcription of stress genes and as a consequence, to interact with biological membranes with alteration of their relative physical state. Such compounds and corresponding pharmaceutically acceptable derivatives and/or salts have applications in the areas of pharmaceuticals, more specifically in cosmetics and dermatology, for all those afections related to an alteration of the expression of stress genes.

Terms Used in the Following Description of the Invention

Aglicons The aglicons are those compounds that in the present invention bind sugars residues (e.g. glucose, fucose, xylose, etc.) forming glycosides. If the sugar moiety is made of by one or more molecules of glucose, such compounds are also defined as glucosides. In a glycoside the non-sugary moiety is defined as “agliconic portion”. Aglicons and glycosides usually have names recalling the natural source from which they have been isolated for the first time.

Gene expression This term designates a mechanism by which an organism synthesizes a protein coded by a specific gene by accumulating an intermediate mRNA.

Heat shock genes (stress genes): ubiquitarious genes that are rapidly transcriptionally activated when cells are exposed to a sudden increase in temperature and/or to various forms of stresses. Stress inducibility is determined by the presence of specific cis elements in the promoter region of this genes (e.g. heat shock element, HSE).

Gene Reporter are genes whose proteic product is easily measured. They are used to analyze and determine the regulating zones of promoters of specific genes (cis sequences). They are used under the control of a promoter of which the transcriptional activity is to be tested.

L929 cell line Cell line of fibroblasts of murine fibrosarcoma.

MPS: Membrane physical state. In the following description the physical state is intended to comprise also the dynamic state, even when not expressely mentioned.

Membrane: semi-permeable barrier that surrounds eukaryotic and prokaryotic cells, organelles (e.g. mitochondria, chloroplasts, endoplasmic reticulum, nuclei, etc), that is composed by a lipid bilayer in which intrinsic membrane proteins or associated proteins are present, and in some cases, cholesterol, ergosterol or glycolipids. All membrane, at different levels among them, undergo cell specific changes in their physical state as a result of the activity of the molecules of the present invention.

Heat shock proteins (HSPs or stress proteins): the protein product of heat shock genes rapidly accumulated by a cell after exposure to stress and whose functions include: assign the proper folding of nascent polypeptides, targeting of denatured proteins (misfolded), protection of mitochondrial and chloroplasts functions, mRNA maturation, their insertion in membrane to protect MPS, etc.

Integral (or intrinsic) membrane proteins: Any membrane protein that, partially or totally, interacts with the hydrophobic region of the phospholipid bilayer and that can be extracted from membrane only by detergents.

PCR (polymerase chain reaction): technique to synthesize in vitro large amounts of specific nucleotide sequences by the use of specific oligonucleotide primers complementary to sequences of the target gene using special termostable DNA polymerases.

Promotor: a specific DNA region onto which RNA polymerase initiates mRNA transcription. The promoter includes a site for DNA binding recognition.

Signaling transduction pathways: Conversion of a signal from a physical (e.g. or temperature, osmolarity) chemical (e.g. hormones) form into an other. In cell biology, this term is referred to the sequential process initiated by the interaction of a chemical factor with a membrane or cell receptor or a physical effect on membrane that culminates in one or more specific cell response (e.g. gene transcriptional activation of sequences under this control).

Transformation: method to obtain proteins through DNA recombinant techniques that requires the cloning of a gene coding for a given protein and where “cloning” means isolation, purification and sequencing of the gene coding for that protein. Once cloned, the nucleotide sequence can be inserted in an appropriate expression vector and the obtained DNA recombinant molecules can be introduced in a microorganism in which the gene is simultaneously replicated with the host DNA. The gene can eventually be re-isolated with standard techniques of molecular biology.

Cloning vector: DNA molecules that contain the entire genetic information that allows them to replicate when transfected in a host.

Membrane fluidity. A widely used but subjective term that describes the relative diffusional motion of molecules within membranes. Fluidity is used rather than viscosity, because membranes are planar, asymmetric structures, and their properties are not comparable to bulk phases. The term fluidity is meant to convey the impression of lateral diffusion, molecular wobbling and chain flexing, that are found in functional membranes where the lipids are in the fluid-crystalline lamellar phase.

Membrane order. The motional movement of molecules or molecular domains within the membrane. Membrane order can be quantified by estimating the motion of paramagnetic probes and calculating an order parameter from the ESR or NMR spectrum.

Non-lamellar phases. Non-bilayer arrangements of lipids in aqueous media. These can be hexagonal (H_{I}) or inverted hexagonal (H_{II}) arrangements; H_{I} phase is seldom found in membranes.

### BACKGROUND OF THE INVENTION

The Heat Shock Response, or stress response, is one of the better studied homeostatic cell responses, mainly involved in the maintenance of cell functionality in response to diverse environmental stresses and/or in pathologic states (Lindquist. 1986). Such response is mediated by a rapid increase in the transcription of those genes that codify for the stress proteins (Morimoto et al. 1998). It has been abundantly demonstrated that such increase in mRNA synthesis of stress genes, and the relative intracellular accumulation of HSPs, are associated with the acquisition of thermotolerance, with protection to subsequent exposure to other forms of stresses or in pathological conditions, etc. (Singer & Lindquist 1998; van Eden & Young 1996; Morimoto et al, 1998). It has been demonstrated that the primary sensor(s) of temperature variations, and in general to other forms of stresses, is (are) localized in the membrane (Carratù et al 1996; Horvath et al 1998, Vigh & Maresca, 1998; Suzuki et al 2000, Piper et al 2000; Torok et al 2001). Further, recent studies have shown that an abrupt temperature change or exposure to other forms of stress, determine a physical re-organization of lipid and protein membrane components (Slater et al 1994), that is followed by a specific gene response aimed to compensate variations in MPS. Thus, a cross-talk between changes in MPS and regulation of gene expression exists, particularly for heat shock genes.

Among the agents responsible of an appropriate MPS we mention desaturases that through their enzymatic activities control the membrane phospholipid composition. Desaturases are enzymes that introduce double bonds in saturated fatty acids (SFA) transforming them into unsaturated fatty acids (UFA). The SFA/UFA ratio is one of the main factors that determines an appropriate MPS in all cells (Cossins, 1994). Recently, it has been demonstrated that the inducible synthesis of stress proteins is controlled by a rapid and local variations of several factors:

the membrane lipid composition

membrane lipid/protein interactions

lipid dynamics (MPS changes) (Vigh et al, 1998).

Thus, the MPS changes in stress conditions re-determines the threshold at which HSPs are normally synthesized.

The aim of this invention is to use in cosmetics and pharmacology the properties of some molecules to accumulate endogenous stress proteins. The cosmetic and therapeutic effects are based on the capacity of such molecules to stimulate such molecules that in turn induce intrinsic cellular homeostatic mechanisms that are altered in specific human and animal pathological conditions as well as in the plants. Further, accumulation of stress proteins, whose capacity to induce cell and tissue protection is well known (Edwards et al, 1999; Latchman 1998; Santoro 2000), confers in a specific manner protection from UV exposure, retards aging, protects from environmental stress (e.g. abrupt increase in temperature, dehydration, etc. (van Eden W et al 1996). Therefore, it has been suggested that these molecules by stimulating HS protein synthesis, particularly in the skin, may be utilized as pharmaceutical drugs and as cosmetics. Stress proteins are also involved in the mechanisms of wound healing, in dermatological diseases such as psoriasis (Edwards et al 1999).

It also known that accumulation of stress proteins is altered in several human chronic diseases such as diabetes, degenerative diseases, such as in the central nervous system, in cancer, in inflammation, in rheumatoid arthritis, in wound healing, in autoimmune diseases, in heart diseases, during aging, etc. (Hightower et al, 2000; Polla 1998; Laplante et al 1998; van Eden W et al 1996; Feige et al 1996; Maytin 1992). It has also been reported that pre-induction of stress proteins acts in a protective manner in several clinical diseases. For example, brief episodes of ischemia, that induce the preferential accumulation of HSP72, protect myocardium from subsequent otherwise lethal ischemia (Sammut et al 2001; Marber et al 1995). Furthermore, HSP70 reduces the size of the infarct following ischemia (Okubo et al 2001). The over expression of rat hsp70 gene in transgenic mice increases protection from cardiac and cerebral ischemia (Rajdev et al 2000; Plumier et al 1996; Plumier et al 1995). The main pre-requisite of HSP inducible drugs is that they must be non toxic and lack side effects together with the property to mimic the effects of stressing agents or, in the absence of stress or in limited stress or in altered physiological conditions of cell targets, to lower the threshold of stress condition in such a way that signals that induce cascade effects are initiated and that cause the transcriptional induction of stress genes. Several agents that induce heat shook protein accumulation have been identified. However, so far, the only one reported to be non-toxic is bimoclomol™. We have now identified a family of chemical compounds of plant origin, particularly useful to be used to modify MPS, thus inducing an increase in amount of synthesis of stress proteins.

### SUMMARY OF THE INVENTION

There are objects of the present invention flavonoid compounds of a general formula (I) and (II) that can be used in the pharmaceutical field, particularly in cosmetics, to modify the cell membrane physical state, particularly by increasing the synthesis of stress proteins.

Further objects of the invention are the pharmaceutical compositions comprising, as active principle, the molecules of the general formula (I) and (II) and relative mixtures.

Further object of the present invention is the chemical synthesis to obtain the molecules of the general formula (I) and (II).

Further object of the present invention is the use of molecules of the general formula (I) and/or (II) to treat pathological conditions derived from an alteration of membrane physical state of eukaryotic cells, of plant cells, of animal cells, particularly, mammalian and human cells, with lack of toxicity and/or side effects. Further objects of the invention are: a method to modify MPS, method to induce stress response, such as heat shock, a method to induce cell protection in eukaryotic cells (e.g. L929, human keratynocytes, etc.) by treatment of cells, tissue or entire animal or plant with an effective amount of the compounds of the general formula (I) and/or (II) and corresponding pharmaceutically effective derivatives and/or salts, including optically active molecules and relative mixtures. Either L929 cells or keratynocytes are preferentially transfected with luciferase genes whose expression is under the control of a human hsp70 promoter.

A further object is a method for the prevention and/or treatment of related alterations connected with modification of cell membrane physical state in plants as well as in animal cells, particularly human.

There are also objects of the present invention the flavonoid products obtained by extraction and purification from vegetable material that contain them, utilizable according to the invention, to modify MPS and induce heat shock transcriptional activation.

Other objects will be evident from the detailed description of the invention.

### BRIEF DESCRIPTION OF THE DRAWINGS

FIG. 1. Plasmid vector pGL3 containing luciferase as a reporter gene under the control of the heat shock promoter (HSE element). Further, the vector contains the ampicillin and geneticin resistant genes as selectable markers.

FIG. 2. Luciferase assay in L929 cells grown at 37° C., treated with different molecules and in heat shock conditions at 40° C. and 41° C.

FIG. 3. Test to evaluate changes of MPS in artificial membranes (LUVs, Large Unilamellar Vesicles), made of di-oleil-fosfatidyl-ethanolamine, di-oleil-fosfatidyl-choline, cardiolipin and fosfatidylserine, that mimic biological membrane lipid composition. Fluidity has been measured with DPH (1,6-difenyl-1,3,5-esatriene) measuring fluorescence. In the figure the experimental data of molecules #11 and #100 are reported. Molecule #11 increases fluidity (destabilizes membranes) while #100 rigidifies membranes.

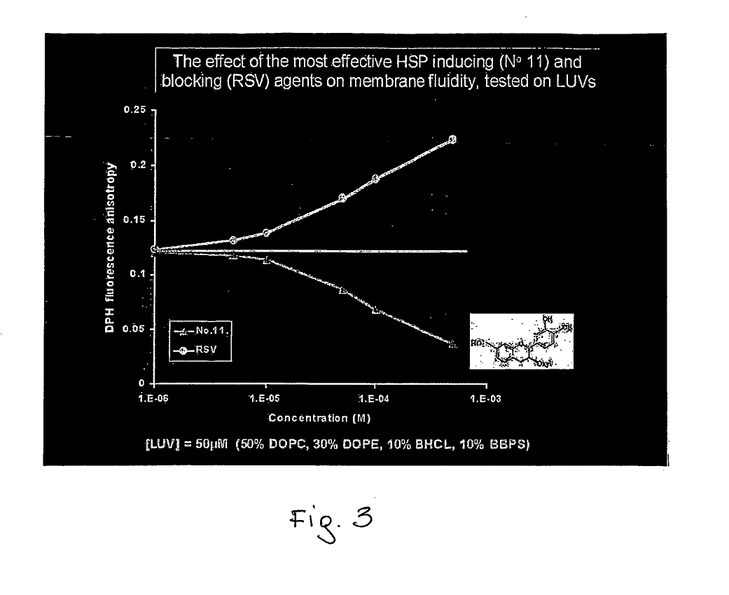

### DETAILED DESCRIPTION OF INVENTION

The present invention is based, at least in part, on the unexpected finding that flavonoid compounds can modify, increasing, the synthesis of stress proteins, as a consequence of the change in MPS that they induce. This finding is significant in the light of the role that HSPs have in the protection of cells from the pathological effects of several diseases. The molecules of the invention are believed to increase stress protein concentration and to protect cells from the side effects of degenerative diseases, such as: tissutal damages, nerve conductivity, membrane cell damage, etc. The molecules of the invention are particularly active in inducing the synthesis of stress proteins such as HSP70, HSP72, HSP90 etc. and small heat shock proteins such as HSP17, HSP20, etc. The compounds according to this invention have the general formula (I) and (II) and both belong to the flavonoid family:

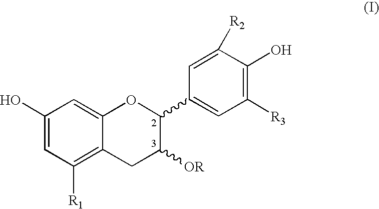

The compounds represented by the general formula (I) are derivatives of flavonoids in which:

R═H, gallate, glycosidic moiety having a number of sugar residues ranging between 1 and 2 equal or different to each other, preferably selected in the group of: β-D-glucose, β-D-mannose, β-D-galactose, β-D-xylose, α-L-arabinose, β-D-quinovose, β-D-fucose, α-L-ramnose, and corresponding mixtures;

R1, R2, R3, equal or different among each other are H or OH.

There are within the scope of this invention the peracetylate derivatives of the compound having formula (I), to say compounds in which OH groups are esterified with acetic acid. They represent important intermediates in the synthesis of the molecules of this invention.

The C atoms in positions (2) and (3) may have configuration R and S. Molecules of the general formula (I) have two chiral centers, in C 2 and 3, with the possibility to produce 4 different diastereoisomers (different combinations of configurations): [2R,3S], [2R,3R], [2S,3R], [2S,3S]. Starting from the observation that in nature the configuration 2R is largely diffused for biogenetic reasons and that glycoside derivatives have a higher biological activity in the reported molecular and biophysical assays, such molecules ere considered more attractive and their synthesis is afterward reported.

Preferred molecules according to the general formula (I) are indicated with the following general formula: derivative of (+)-catechin [2R, 3S]

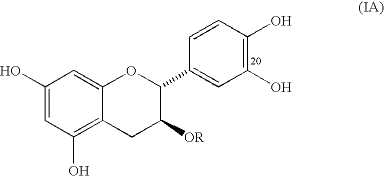

in which R is selected in the group of the following substituents:

R═H; R=β-D-glucose^{*.#(Sasuga et al. 2000, Bae et al. 1994)}; R=β-D-mannose^{#(Sasuga et al. 2000)};

R=β-D-galactose^{#(Sasuga et al. 2000)}; R=β-D-xylose^{#(Sasuga et al. 2000)}; R=α-L-arabinose;

R=β-D-quinovose; R=α-L-ramnose^{* (Banefeld et al. 1986)}; R=gallate^{§}

Derivatives of (−)-epicatechin [2R, 3R]

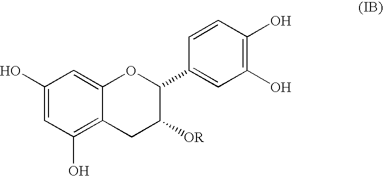

in which R is selected in the group of the following substituents:

R═H; R=β-D-glucose^{*(Modmoto et al. 1986)}; R=β-D-mannose; Rβ-D-galactose; R=β-D-xylose; R=α-L-arabinose; R=β-D-quinovose; R=α-L-ramnose; R=gallate^{§}

Derivatives of (+)-fisetidinol [2R, 3S]

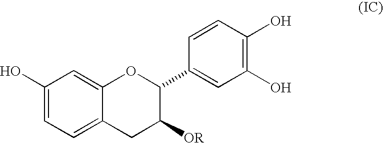

in which R is selected in the group of the following substituents:

R═H; R=β-D-glucose; R=β-D-mannose; R=β-D-galactose; R=β-D-xylose^{*(Placente et al. 1999)}; R=α-L-arabinose; R=β-D-quinovose; R=α-L-ramnose; R=gallate

Derivatives of (+)-guibourtinidol [2R, 3S]

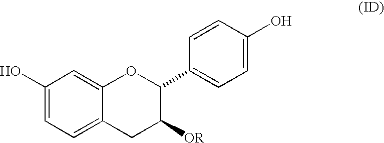

in which R is selected in the group of the following substituents: R═H; R=β-D-glucose; R=β-D-xylose; R=β-D-quinovose

Derivatives of (+)-efzelechin [2R, 3S]

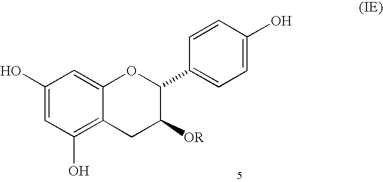

in which R is selected in the group of the following substituents: R═H; R=β-D-glucose; R=β-D-xylose; R=β-D-quinovose

Derivatives of flavan-3-olo with 2S Stereochemistry

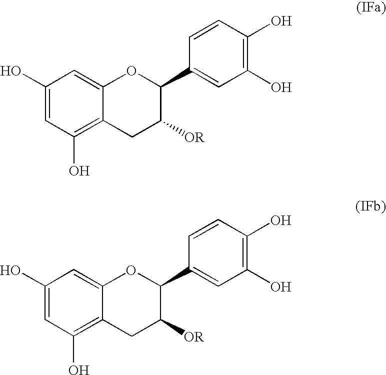

in which R is selected in the group of the following substitutive groups respectively:

(IFa) (IFb)

R═H (−)-catechin [2S, 3R] R═H (+)-epicatechin [2S, 3S]

R=gallate^{§} R=gallate^{§}

Derivatives of (−)-epigallocatechin [2R, 3R]

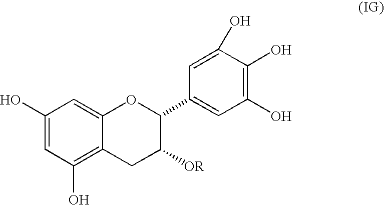

in which R is selected in the group of the following substituents: R═H; R=gallate^{§}

In the above mentioned molecules the apici have the following meanings:

*natural compound; #synthetic molecule;

§molecule commercially available

Further, bibliographic references in which they are described are also reported. The molecules represented by the general formula (II) are flavonic e flavonolic derivatives.

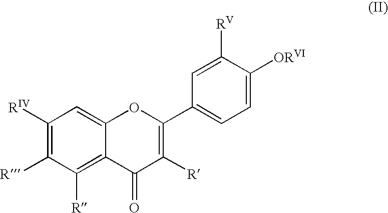

In which:

R′=H, OH, O-glycosidic portion that has a number of sugar residues ranging between 1 and 2, equal or different among them and bound each other, preferably chosen among β-D-glucose, β-D-galactose, β-D-xylose, α-L-ramnose, and corresponding mixtures;

R″═H, OH; R′″═H, OH, C-glucose; R^{IV}═H, OMe, O-glycosidic portion that has a number of sugar residues ranging between 1 and 2, equal or different among them and bound each other, preferably chosen among β-D-glucose, β-D-galactose, β-D-xylose, α-L-ramnose, and corresponding mixtures R^{V}═H, OH R^{VI}═H, β-D-glucose.

Preferred molecules according to the general formula (II) are indicated with the following general formulas:

Derivative of quercetin

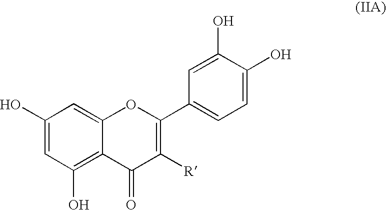

in which R′ is chosen among one of the following substituents:

R′═OH, O-β-D-glucose, O-β-D-galactose, O-β-D-xylose, O-α-L-ramnose, O-β-D-glucose 6→1-α-L-ramnose (the arrow between the two units of glucose shows the binding with the next sugar)

Derivative of canferol:

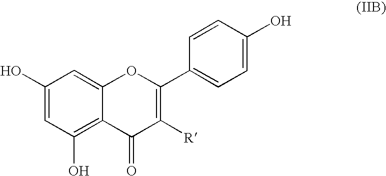

in which R is chosen between one of the following substituents:

R′═OH, O-β-D-glucose, O-β-D-galactose, O-β-D-xylose, O-α-L-ramnose, O-β-D-glucose 6→1-α-L-ramnose (the arrow between the two units of glucose shows the binding site with the next sugar).

Derivative of luteolin

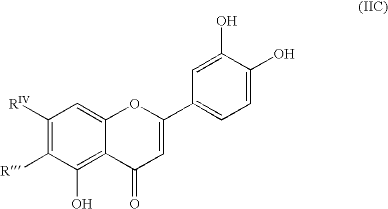

In which: R′″═H, OH, C-β-D-glucose, C-β-D-glucose-2→1-O-α-L-ramnose R^{IV}═OH, O-β-D-glucose

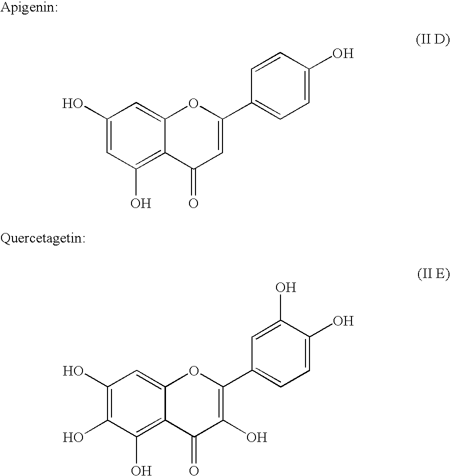

Derivative of fisetin

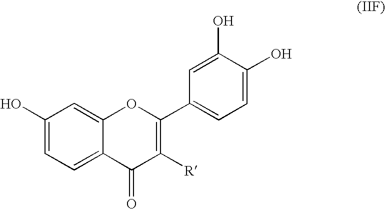

in which R′ is chosen among one of the following substituents:

R′═OH, O-β-D-glucose, O-β-D-galactose, O-β-D-xylose, O-α-L-ramnose.

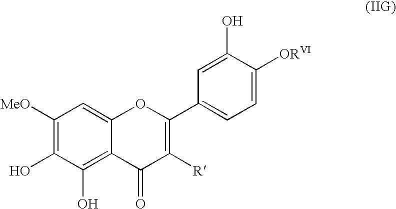

Preferred molecules according to the formula (IIG) are molecules in which:

R′═OH, R^{VI}═H;

R′═O-β-D-glucose, R^{VI}═H;

R′═OH, R^{VI}=β-D-glucose;

R′═O-(β-D-glucose1→4-O-β-D-glucose) (the arrow between the two units of glucose shows the binding site with the next sugar), R^{VI}═H;

R′═O-(α-L-ramnose1→2-O-β-D-glucose) (the arrow between the two units of glucose shows the binding site with the next sugar), R^{VI}═H;

R′═O-[(2-caffeoil)-β-D-glucose1→4-O-β-D-glucuronic] (the arrow between the two glucosidic units shows the binding site with the next sugar), R^{VI}═H.

All compounds specified, in their diastereoisomers and/or optically active pure forms and their relative mixture are part of this invention.

The molecules of the general formulas (I) and (II) may be synthesized, for example, starting from the corresponding flavonoidic aglicons according to standard procedures of organic chemistry or can be purified from plants as, e.g., Anadenanthera macrocarpa, Potentilla viscosa, Calliandra haematocephala, Guibourtia coleosperma, Paepalanthus latipes and Paepalanthus velloizioides with standard extraction procedures or can be obtained commercially,

There are also included in the present invention the flavonoidic compounds extracted from plants, algae and sea weeds that contain them. Such extraction products can also be utilized according to the aim of this invention to modify MPS and induce stress genes. They can be obtained by using standard procedures and generally are flavonoid mixtures that can be used as such or following different steps of purification to separate more pure compounds that have particular biological interest.

Regarding molecules of formula (I), some of them, particularly aglicons (+)-catechin (2R,3S) and (−)-epicatechin (2R,3R), can be isolated from several plants or commercially available (e.g. Sigma) as starting material for related products. For example, the green tea (Camelia sinensis) is the main source of (−)-catechin (2S,3R), (−)-catechin-3-gallate (2S,3R), (+)-epicatechin (2S,3S), (−)-epicatechin-3-gallate (2R,3R). On the other hand, only occasionally efzelechin (2R,3S), fisetinidol (2R,3S) and guibourtinidol (2R,3S) are isolated from natural sources. Particularly, molecules of formula (II), can be isolated from the following Brazilian plants: Paepalanthus latipes and Paepalanthus velloizioides (Eriocaulaceae) (Vilegas et al 1999), as indicated in the examples.

The extraction can be performed on vegetable material, better if first dried. Several extraction steps are performed with solvents such as ether, chloroform, methanol, water and corresponding mixtures, that are later removed, generally by evaporation. The extracted material, redissolved in an appropriate solvent, is further fractionated by column chromatography. The eluted products are collected and characterized.

A general procedure of synthesis that can be used to prepare aglicons from flavan-3-oli includes the dioxydrilation of 1,3-diarilpropen, followed by acid-catalyzed cyclization, that produces diastereoisomers, according to procedures reported in the literature (Scheme 1), Nel et al. (1999). The hydrogenation of calcon (1) (E)-1-(4′-O-metoxymetylfenyl)-3-(2″,4″-di-O-metoxymetyl-fenyl)-propenon in the presence of Pd/C produces retro-dihydrocalcon (2) that, after reduction with NaBH₄, produces 1,3-diarilpropan-1-olo (3) that is converted in (E)-1,3-diarilpropen (4) using SOCl₂ and 1,8-diazabicyclo[5.4.0]undec-7-ene (1,8-DBU). From compound (4) by shaking in difasic system BuOH:H₂O 1:1 sin-diol is produced (5) by treatment with MeSO₂NH₂ at 0° C. that, after deprotection and cyclization, produces the flavanic derivative (6).

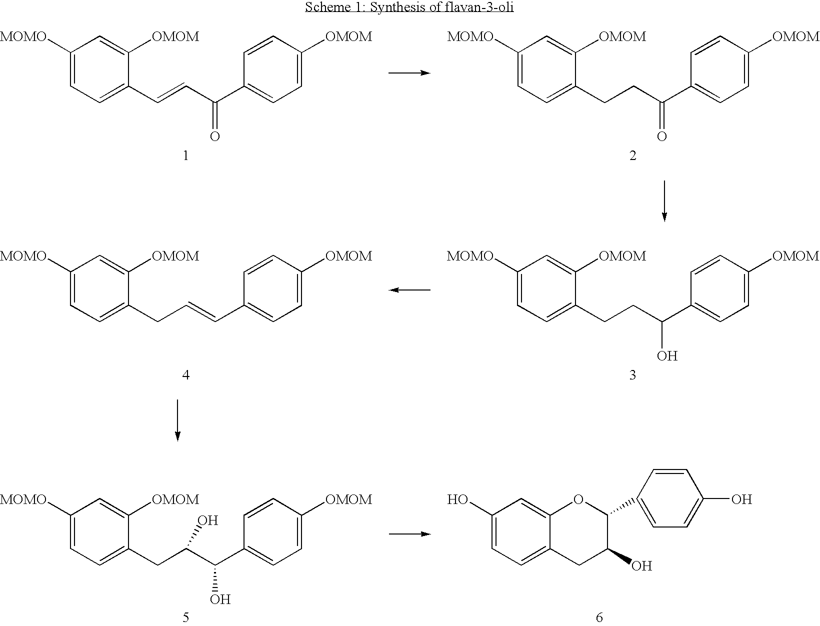

Flavan-3-olo, acetylated in the aromatic —OH according to known organic reactions, can interact with the bromide of the peracetylated sugar to produce the corresponding glycoside.

A general procedure of synthesis that can be used to produce flavan-3-O-glycosids includes the initial synthesis of the appropriate aglicon in the aromatic —OH followed by the reaction of this with the halide of the sugar that had previously peracetylated. The so obtained compound is then desacetylated (scheme 2).

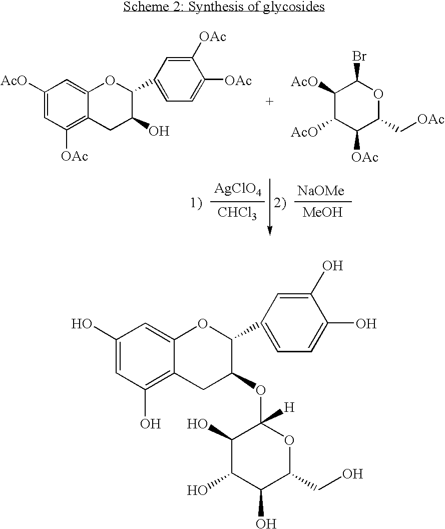

The molecules of formula (I) and (II) according to this invention and the corresponding pharmacologically acceptable salts and/or derivative, and the corresponding molecules in their diastereolsomer and/or optically active pure forms and corresponding mixtures, can be used in pharmaceutical applications, particularly in dermatology and cosmetics. It has been observed that such molecules have biological activity on membranes modifying their physical state. Thus, such compounds can be active in all those clinical diseases that are established when the MPS is altered and membranes are less functional under stress condition: oxidative stress, mechanic stress, osmotic stress, stress due to hypoxia, ischemia, heat shock, radiation shock, shock produced by toxic compounds and free radicals, in degenerative chronic diseases and in the protection from cardiac and cerebral ischemia.

Thus, according to a characteristic of this invention compounds of the formula (I) and (II) can be used to treat: chronic degenerative illness, cardiac cerebral ischemia, diabetes, vascular and cardiovascular diseases, coronary and cerebral diseases, in allergies, immune and autoimmune diseases, of viral or bacterial origin, tumors, skin, mucosal, epithelial, renal diseases, traumas, neurodegenerative diseases, dementia, Alzheimer, Parkinson, epilepsy, AIDS, physiological stresses, ulcers, dermatitis, psoriasis, burns, etc.

According to an other characteristic of this invention compounds of formula (I) and (II) can be used to modify MPS of eukaryotic cells, particularly of animal, plant cells and of microorganisms and in particular of higher organisms and of human, with preventive and therapeutic uses.

Therefore, it is an aspect of this invention a method to modify MPS and to induce heat shock gene transcription including the treatment of the same cells with pharmaceutically acceptable amounts of the compounds of this invention.

Animal, mammalian and plant cells, or of microorganisms, are exposed to heat shock for different length of time (from 5 min to 2 hours or more) and at the same time or subsequently treated with the compounds of this invention. Such treatment causes a change in the MPS of membrane and accumulation of stress proteins.

Alternatively, the treatment includes exposure to the compounds of this invention and treatment with heat shock.

Preferably, treated cells are eukaryotic cells of plant or animal sources, in particular of mammals, more specifically human.

An evaluation of the biological activity of the compounds of the invention can be performed as follows.

The molecular assay according to this invention has been performed using described techniques of molecular biology as for example described in Sambrook et al. (2001), using suitable vectors harboring promoters that can express reporter gene(s) of interest (e.g. a human hsp70 promoter) after exposure to stress (e.g. heat shock in mammalian or human cells, fibroblasts and/or keratinocytes). The identified substances are capable to induce hsp70 gene transcription. Further, the method includes active molecules capable to modify MPS of the same cells or of artificial lipidic membrane. It is thus possible to test their cosmetic effects, dermatological and pharmacological effects of the molecules under test in animal models and human clinical trials.

The molecular method used in this invention can be sketched in the following main steps:

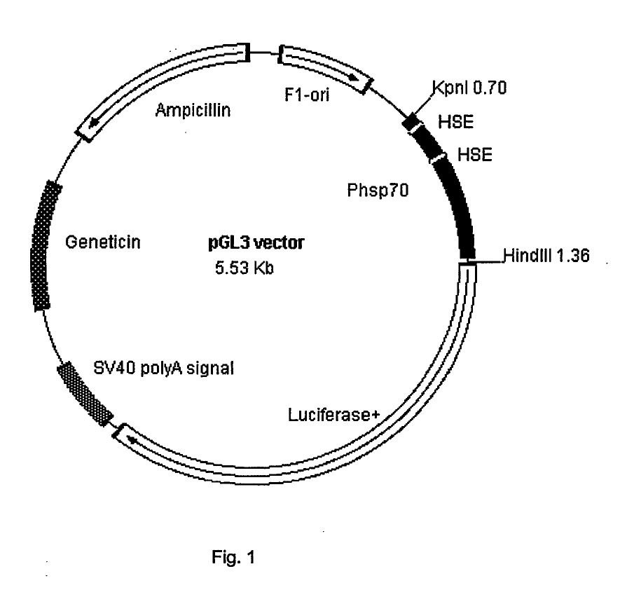

preparation of a suitable vector (e.g. that described in FIG. 1) that harbors the reporter gene coding for a luciferase (or GFP, green fluorescent protein) under the control of an inducible hsp70 promoter by heat shock in mammal or human;

genetic transformation of mammalian or human cell lines with these constructs;

assay of the protein product (luciferase or determination of GFP fluorescence) after heat shock;

determination of anisotropy of the cell lines to determine changes in the MPS of the same cells or in artificial membranes.

Alternatively, rather than using a reporter gene, it is possible to use transcription of heat shock gene by Northern blot directly measuring the increase in hsp70 mRNA accumulation, or by quantitative PCR.

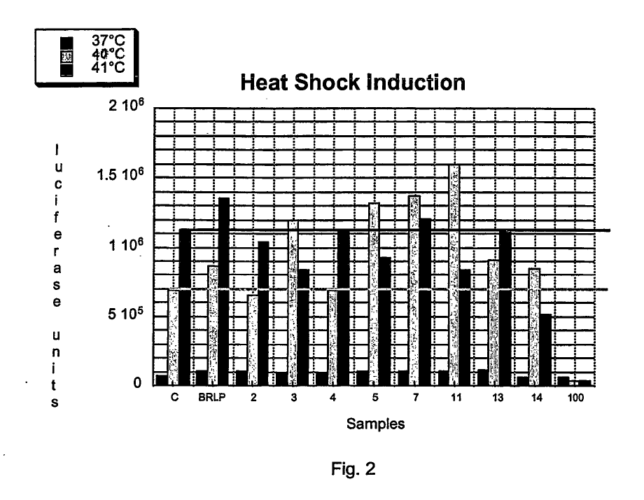

For example, L929 cells, incubated at 37° C., are stressed by heat shock at 40° or 41° C. with exposures variable from 20 min to an hour or more. After heat shock, a molecular assay that involves mRNA purification, its separation on agarose or acrylamide gels followed by hybridization with a labeled probe (e.g. hsp70, hsp17 etc.) is performed. Alternatively, the activity of luciferase used as reported gene under the control of a heat shock promoter can be used (FIG. 1). In this case, after heat shock cells are lysed in the presence of an appropriate substrate for luciferase, the activity is measured with a luminometer. We have identified and purified several molecules (listed in Table 1), from plants or chemically produced as described earlier, that are capable to induce a heat shock response higher or equal to that of Bimoclomol™ (utilized as an internal positive control, FIG. 2).

The test utilized to assay the over expression of heat shock genes is based on a rapid enzymatic assay to determine luminescence of eukaryotic cells transfected with luciferase gene. The luciferase gene has been cloned in an eukaryotic plasmid under the control of human hsp70 gene and transfected in murine fibroblasts (L929 cell line). In such cells, a heat shock induces hsp70 promoter that activates transcription of the downstream gene (reporter gene, e.g. luciferase) whose activity is measured determining luciferase in the presence of luciferine with a luminometer. L929 cells are treated with each of the molecules listed in FIG. 2 at a concentration of 10 μM and immediately exposed to a heat shock at different temperatures. After lysis, luciferase activity has been determined measuring the quantity of light emitted with a luminometer. With this assay it is possible to analyze rapidly the potential activity of several molecules. An additional negative control was established with molecule #100 (resveratrol) that inhibits the inducibility of hsp70 mRNA transcription by heat shock. Similar assays measuring hsp70 mRNA transcription was induced by heat shock, in constructs in which the luciferase gene had not been cloned, as a result of the exposure to the mentioned molecules can be established and measure by Northern blot.

All the molecules with heat shock mRNA inducibility are under the control of hsp70 promoter (or to inhibit) have been evaluated in relation to their capacity to perturb MPS.

The assay that shows the capacity to modify membrane fluidity has been performed on artificial membranes (LUVs), made of di-oleil-fosfatidil-ethanolamine, di-oleil-fosfatidil-coline, cardiolipin and fosfatidylserine, that mimic the membrane lipid composition. In these membranes, every 500 lipid molecules is inserted. 1 molecule of DPH This substance emits fluorescent light when it is excited with polarized light. The more a membrane is rigid the less is the capacity of the molecules to rotate freely in the membrane. Using a fluorimeter that detects the emitted fluorescence by DPH, either vertically in respect to the polarizing light or horizontally, the anisotropy of the membrane is determined and correlated to its fluidity. The more the membrane is fluid, the more DPH rotates freely and emits light in a direction different from that with which it receives the excitation. To a higher value of anisotropy corresponds a higher rigidity of the membrane, while a lower anisotropy (low fluorescence) corresponds a higher fluidity. The example with molecule #11 (IC containing-β-D-xylose in position 3 that is anadentoside, (+)-fisetidinol [2R,3S] 3-β-D-xylose) and with #100 (resveratrol) show that molecules that fluidify biological membranes also induce a heat shock response, while a membrane rigidification corresponds inhibition of heat shock genes, or of genes controlled by a heat shock promoter.

A further aspect of this invention is the preparation of pharmaceutical compositions that include molecules of formula (I) and (II), either optically active and/or containing diastereoisomerically pure molecules or in mixtures, as salts and/or as derivatives, all of them pharmaceutically active, that can be easily synthesized by the expert in the field. These compositions can be prepared by known methodologies, by mixing the active principle preferably in a concentration between 0.1 and 99.5% in weight with other components. The other components of the mixture can advantageously contain, also in combination, non active ingredients such as: eccipients, diluents, stabilizers, or other adjuvants such as to obtain compositions administrable orally, parenterally, rectaly, topically, spray. For example, as pills, tablets, capsules, granules, syrup, solutions, suspensions, creams, ointments, gels, powders, controlled or retarted formulations.

The kind of mode of administrations and dosage and quantities will depend on the type of disease and kind of formula used. The composition of creams for topical use in cosmetics are particularly preferred. They can be prepared according to known techniques that mix the active principle(s) with other ingredients.

The following examples are presented to show the invention and are not to be considered limitative of its scope.

Materials and Methods

The chemicals used were pure products by Aldrich, Fluka, Carlo Erba, Sigma, Stratagene, Clontech, Amersham, etc.

The instruments used are routinely used in chemistry, analyses of molecules, DNA sequencer, NMR, luminometer, spectrophotometers, electrophoresis apparatuses, etc

### EXAMPLE 1

### Synthesis of Guibourtinidol (2R,3S, Molecules ID) and its Diastereomers

The synthesis (as referred to scheme 1) involves the following steps: condensation (performed according to Nel et al. 1999) of 2,4-di-O-metoxymethylbenzaldehyde and 4-O-metoxymethylaceto-fenone with yields of ca. 70% producing (E)-retro-2,4,4′-tri-O-metoxymethylcalcone (1).

The quantitative hydrogenation of (1) in presence of 5% carbon palladiate produces (2). Reduction of (2) with Na BH₄ synthesizes 1,3-diarylpropane-1-olo (3), converted into (E)-1,3-diarylpropene (4) with a yield of ca. 60% using SOCl₂ and 1,8-DBU (1,8-diazobicycl [5.4.0] indec-7-ene). Treatment of (4) with MeSO₂NH₂ in a biphasic system BuOH/H₂O (1:1) produces 1S,2S-sin-diolo (5) with a yield of ca. 70%. Simultaneous deprotection and cyclization of (5) with 3 M HCl in methanol at 60° C. produces (2R,3S)-2,3-trans4′,7-diidroxyflavan-3-olo (6) (60% yield) and (2S,3S)-2,3-cis-4′,7-diidroxyflavan-3-olo (7) (20% yield).

### EXAMPLE 2

### Compound IA: (+)-Catechin

(+)-catechin (and/or (−)-epicatechin) acetylated with acetic anhydride in pyridine produces 3′,4′,5,7-tetra-O-acetyl-(+)-catechin (and 3′,4′,5,7-tetra-O-acetyl-(−)-epicatechin respectively) that, treated with tetra-O-acetyl-β-D-glucopyranosilbromide, produces 3-O-β-D-glucopyranoside peracetylated (scheme 2). The latter, after saponification with sodium metoxyde in methanol, generates respectively (+)-catechin-3-O-β-D-glucopyranoside (or Ia(−)-epicatechin-3-O-β-D-glucopyranoside). The yields of the reaction can be increased utilizing the procedure for the synthesis as described by Sasuga et al 2000, using silver perchlorate and silver trifluoromethansulfonate as condensing agent.

### EXAMPLE 3

### Extraction of fisetinidol-3-O-β-D-xylopiranoside (Anadentoside)

Fisetinidol-3-O-β-D-xylopiranoside has been isolated by the bark of Anadenanthera macrocarpa (Leguminosae), a South Americanan vegetable species (Bolivia) (Piacente et al 1999).

The dried vegetable material (ca. 300 gr) has been initially extracted with ether and then with chloroform (1.4 gr). The same material then has been extracted with a mixture chloroform-methanol 9:1 (4.0 gr). The extracted material, dissolved in methanol, has then been fractionated on a Sephadex LH-20 column (Pharmacia). Collecting fractions of ca. 10 ml, fractions 50-58 contained 25 mg of compound IC with R=β-D-xylose. The latter has been characterized by nuclear magnetic resonance and mass spectrometry as reported by Piacente et al 1999.

### EXAMPLE 4

### Characterization of Compound fisetinidol-3-O-β-D-xylopiranoside.

The molecular formula (C₂₀H₂₂O₉) was determined with experiments using ¹³C NMR, ¹³C DEPT NMR and FAB-MS in negative ions, the compound showed a quasi-molecular peak [M−H]⁻ at m/z 405 and a fragment at m/z [(M−H)-132]⁻ due to the loss of a pentose unit. The spectrum ¹H NMR in the aromatic region showed signals a δ 6.72 (1 H, dd, J=2.0 and 8.3 Hz, H-6′), 6.76 (1H, d, J=8.3 Hz, H-5′) and 6.82 (1H, d, J=2.0 Hz, H-2′) due to the presence of the ring B 1′,3′,4′-trisubstitued of a flavanoidic skeleton and signals at δ 6.33 (1H, d, J=2.0 Hz, H-8), 6.36 (1H, dd, J=2.0 and 8.3 Hz, H-6) and 6.85 (1H, d, J=8.3 Hz, H-5) in agreement with the presence of a single oxydrilic group at C-7 in the ring A. Also evident were signals at δ 2.82 (1H, dd, J=6.2 and 15.6 Hz) and 2.87 (1H, dd, J=4.8 and 15.6 Hz), typical of H₂-4 of a flavanic derivative and at δ 3.10 (1H, dd, J=7.3 and 8.7Hz), 3.15 (1H, t, J=11.4 Hz), 3.23 (1H, t, J=8.7 Hz), 3.45 (1H, ddd, J=6.2, 8.7 and 11.4 Hz), 3.85 (1H, dd, J=6.2 and 11.4 Hz), 4.15 (1H, m), 4.16 (1H, d, J=7.3 Hz) and 4.97 (1H, d, J=5.9 Hz) all of which that can be attributed to protons with alcoholic functions. The DQF-COSY spectrum showed the CH₂ (δ 2.82 and 2.87)-CHOH (δ 4.15)-CHOH (δ 4.97) sequence due to the presence of an aliphatic eterocyclic ring of a flavanol and the typical system of spin of β-D-xylopyranose. In particular, the coupling constants of the signals that can be attributed to H-2 (J=5.9 Hz) and to H-3 (J=4.8, 5.9 and 6.2 Hz) of the aglicon suggested a C-2 and C-3 with the same stereochemistry of (+)-catechin. The HSQC experiment, that correlates the protonic signals to the corresponding carbonic signals, allowed to establish the presence of a shift due to the glycosidation at C-3 of the aglicon (δ 76.9), allowing to infer that the residue of xylose was bound to C-3. The HMBC spectrum that showed the correlation between the protonic signals at δ 2.82 and 2.87 and C-10 (δ 112.4), C-5 (δ 131.5), C-9 (δ 155.9), the protonic signals at δ 4.15 and C-2 (δ 80.7), between the protonic signals at δ 4.97 and C-1′ (δ 132.2), C-2 (δ 114.8) and C-6′ (δ 119.6) allowed us to assign the resonances of the quaternary carbons and to infer for the aglicon of IC, the structure of 3,3′,4′, 7-tetrahydroxyflavan (fisetinidol). An additional correlation was observable between the signal of the anomeric proton at δ 4.16 and C-3 (δ 76.9) of fisetinidol. On the bases of such data to the compound IC with R=β-D-xylose has been assigned the structure fisetinidol-3-O-β-D-xylopyranoside, called anadantoside.

### EXAMPLE 5

### Isolation of Derivatives of 7-O-methylquercetagetin—General Formula (IIG)

Inflorescences and pulverized leaf powder (100 g in both cases) of P. latipes and P. velloizioides were extracted with chloroform and then with 80% methanol (steeping at room temperature, 1 week for each solvent). Solvents were then vaporized under vacuum, followed by collection of the solid material. The concentrated methanolic extracts of each plant were dissolved in water, filtered and chromatographed on a XAD-2 column with 3 liters of water and then with 1 liter of methanol. An aliquot (ca 1.2 g) of the methanolic extract of P. latipes was separated on a Sephadex LH-20 (80×2 cm). Fractions of .ca 8 ml were eluted with methanol and checked by TLC using as eluant buthanol/acid acetic/water (13:3:5). Fractions 31-36 and 41-44, obtained with the Sephadex, were further purified by HPLC Waters μ-Bondapak RP-18 (30 cm×7.6 mm i.d.) column using methanol/water (9:11) as eluant; from fractions 31-36 we obtained in pure form, compounds with R′=β-D-glucose 1→4 β-D-glucose and R″═H; with R′═β-D-glucose and R″═H; from fractions 41-44 we obtained in pure form compounds con R′=6-caffeoil-β-D-glucose 1→4 β-D-glucoronic and R″═H, fractions 51-56 contained, in pure form, compound R′═H and R″=β-D-glucose and fractions 67-70 contained, pure, compounds R′═R″H; The methanolic extract (1.2 g) of P. velloizioides, was chromatographed on a Sephadex LH-20 and on a HPLC with the previously described conditions; from fractions 33-40 we obtained in pure form, compounds II with R′=α-D-glucose 1→4 β-D-glucose and R″═H; II with R′β-D-glucose and R″═H, from fractions 48-52 compound II with R′=α-L-ramnose 1→2 β-D-glucose and R″═H and from fractions 64-69 compound II with R′═R″═H.

TABLE 1

| #C | Control |
|---|---|
| #BRLP | bimoclomol ™ |
| #2. | quercetagetin-7-methyl ether |
| #3. | quercetagetin-7-methyl ether-4′-O-β-D-glucopyranoside |
| #4. | quercetagetin 7-methyl ether-3-O-neohesperidoside |
| #5. | quercetagetin-7-methyl ether-3-O-β-D-glucopyranoside |
| #7. | 6-idroxyluteolin-7-O-β-D-glucopyranoside, |
| #11. | fisetinidol-3-O-β-D-xylopyranoside |
| #13. | luteolin-6-C-β-D-glucopyranoside |
| #14. | luteolin-6-C-[α-L-ramnopyranosil-(1->2)-O]-β- |
| | D-glucopyranoside |
| #100. | Resveratrol |

### REFERENCES

Bae, Y. S., et al.; Phytochemistry 35, 473-478 (1994)

Biro, K., et al.; G NeuroReport. 9: 2029-2033 (1998)

Banefeld, M., et al.; Phytochemistry 25, 1205-1207 (1986)

Carratu L, et al.; Proc Natl Acad Sci USA 93: 3870-3875 (1996)

Cossins A Temperature adaptation of biological membranes, Edited by Cossin, A. R., London, Portland Press, (1994)

Edwards M J, et al.; J Cutan Pathol 26: 483-489, (1999)

Horvath I, et al.; Proc Natl Acad Sci USA. 95: 3513-3518 (1998)

Kamya S, Nakagawa, Y, Sasuga, K. Japanese Patent CAN: 121: 91330 AN 1994:491330.

Latchman, D S Int. J. Mol. Med. 2: 375-381 (1998)

Lindquist, S. Ann. Rev. Biochem. 55:1151-1191 (1986)

Marber M S, Mestril R, Chi S H, Sayen M R, Yellon D M & Dillmann W H J Clin Invest, 95,1446-56 (1995)

Maytin, E V J Biol Chem 267: 23189-96 (1992)

Morimoto R I and Santoro M G Nat Biotech, 16: 833-38 (1998)

Morimoto, S., et al.; Chem. Pharm. Bull. 34, 633-642 (1986)

Okubo S, Wildner O, Shah M R, Chelliah J C, Hess M L, Kukreja R C Circulation 103:877-81 (2001)

Nel J J Reinier, et al. Phytochemistry 52, 1153-1158 (1999)

Piacente, S., et al.; C. Phytochemistry 51, 709-711 (1999)

Plumier J-C I. & Currie R W Cell Stress & Chaperones 1, 13-17 (1996)

Plumier J-C I., Ross B M, Currie R W, Angelidis C E, Kaziaris H, Kollias G &

Pagoulatos G N J. Clin. Invest. 95, 1854-60 (1995)

Rajdev S, Hara K, Kokubo Y, Mestril R, Dillmann W, Weinstein P R, Sharp F R Ann Neurol 47:782-91 (2000)

Sambrook J, et al.; Molecular Cloning: A Laboratory Manual Third Edition CSH

Laboratory press, Cold Spring harbor, N.Y., (2001)

Sammut I A, Jayakumar J, Latif N, Rothery S, Severs N J, Smolenski R T, Bates T E, Yacoub M H Am J Pathol. 158:1821-31, (2001)

Santoro M G. Biochem Pharmacol. 59: 55-63 (2000).

Sasuga, K., et al.; Chagyo, Kenkyu, Hokoku 89, 29-36 (2000)

Singer M A, et al.; Trends Biotechnol. 16:460-468 (1998)

Slater S J, et al.; J Biol Chem. 269: 4866-4871 (1994)

Suzuki I, et al.; EMBO J. 19: 1327-1334 (2000)

Torok Z, et al.; Proc Natl Acad Sci USA 98: 3098-3103 (2001)

van Eden W et al.; Stress Proteins in Medicine, Eds., Marcel Dekker Inc. (1996)

Vigh L, et al.; Trends Biochem Sci. 23: 369-374 (1998)

Vigh, L., et al.; Nature Medicine 3: 1150-1154 (1997)

Vilegas, W., et al., Phytochemistry 51, 403-409 (1999)

1. Flavonoidic compounds of the general formula (I) and (II): 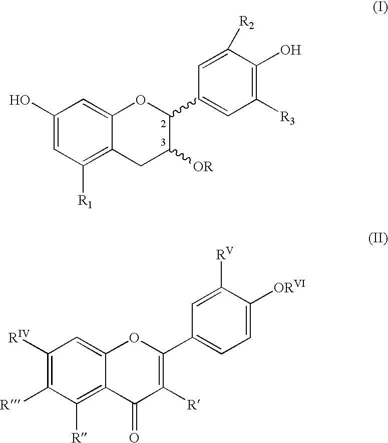
to be used in the pharmaceutical field in which:
R═H. gallate, glicosidic portion having a number of sugar residues, equal or different among each other and bound one to the other, comprised between 1 and 2;
R1, R2, R3, equal or different among each other, are H or OH
R′═H, OH, O-glicosidic moiety carrying a number of sugar residues, equal or different among each other and bound one to the other, comprised between 1 and 2;
R″═H, OH
R′″═H, OH, C-glucose
R^{IV}═H, OMe, O-glicosidic moiety carrying a number of sugar residues, equal or different among each other and bound one to the other, comprised between 1 and 2;
R^{V}═H, OH
R^{VI}═H, β-D-glucose.

2. Flavonoidic compounds of formula (I) according to claim 1 selected in the group of compounds having the following substitutes:
R=β-D-glucose, β-D-mannose, β-D-galactose, β-D-xylose, α-L-arabinose, β-D-quinovose, β-D-fucose, α-L-ramnose, and corresponding mixtures.

3. Flavonoidic compounds of formula (II) according to claim 1 selected in the group of compounds having the following substitutes:
R′=β-D-glucose, β-D-galactose, β-D-xylose, α-L-ramnose, and corresponding mixtures;

4. Flavonoidic compounds of formula (II) according to claim 1 selected in the group of compounds having the following substitutes:
R^{IV}=β-D-glucose, β-D-galactose, β-D-xylose, α-L-ramnose, and corresponding mixtures.

5. Compounds according to claim 1 that are derivatives of (+)-catechin [2R, 3S] according to the general formula (IA) 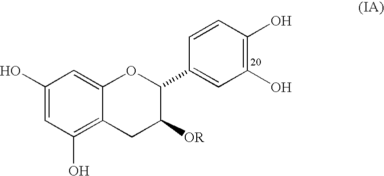
in which R is selected in the group of compounds having the following substitutes:
R═H; R=-D-glucose; R=-D-mannose; R=-D-galactose; R=-D-xylose; R=-L-arabinose; R=-D-quinovose; R=-L-ramnose; R=gallate.

6. Compounds according to claim 1 that are derivatives of (−)-epicatechin [2R, 3R] according to the general formula (IB): 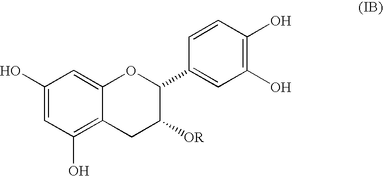
in which R is selected in the group of compounds having the following substitutes:
R═H; R=β-D-glucose; R=β-D-mannose; R=β-D-galactose; R=β-D-xylose;
R=α-L-arabinose; R=β-D-quinovose; R=α-L-ramnose; R=gallate.

7. Compounds according to claim 1 that are derivatives of (+)-fisetidinol [2R, 3S] according the general formula (IC) 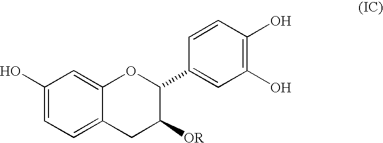
in which R is selected in the group of compounds having the following substitutes:
R═H; R=-D-glucose; R=-D-mannose; R=-D-galactose; R=-D-xylose; R=-L-arabinose; R=-D-quinovose; R=-L-ramnose; R=gallate.

8. Compounds according to claim 1 that are derivative of (+)-guibourtinidol [2R, 3S] according the general formula (ID) 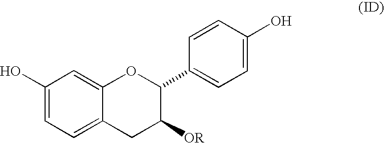
in which R is selected in the group of compounds having the following substitutes:
R═H; R=-D-glucose; R=-D-xylose; R=-D-quinovose.

9. Compounds according to claim 1 that are derivatives of (+)-efzelechin [2R, 3S] according the general formula (IE) 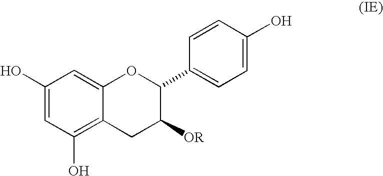
in which R is selected in the group of compounds having the following substitutes:
R═H; R=-D-glucose; R=-D-xylose; R=-D-quinovose.

10. Compounds according to claim 1 that are derivatives of flavan-3-olo with stereochemistry 2S according to the general formula (IFa) and (lFb) 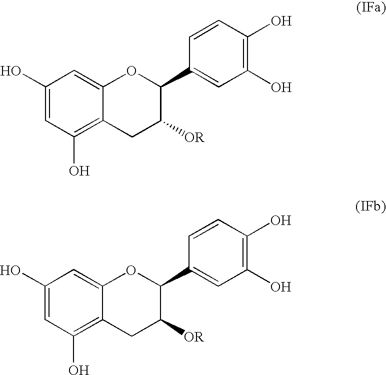
in which R is selected in the group of compounds having the following substitutes of the two following substituents:
R═H (−)-catechin [2S, 3R], R=gallate, according to (IFa);
R═H (+)-epicatechin [2S, 3R], R=gallate, according to (lFb).

11. Compounds according to claim 1 that are derivatives of (−)-epigallocatechin [2R, 3R] according to the general formula (IG) 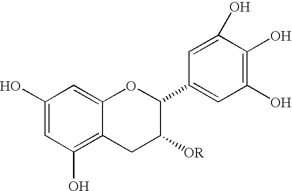
in which R is selected in the group of the following substitutes: R═H; R=gallate

12. Compounds of formula (I) according to claims 1-11 in which the atoms C in position (2) and (3) have the configuration R or S independently from each other.

13. Compounds according to claims 1-12 in which the OH groups are substituted by peracetilyc groups.

14. Compounds according to claim 1 that are derivatives of quercetin according to the general formula (IIA) 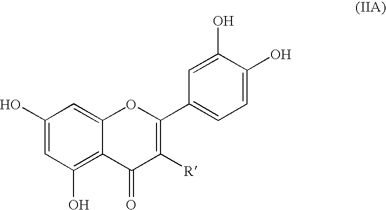
in which R′ is selected in the group of compounds having the following substitutes:
R′═OH, O-β-D-glucose, O-β-D-galactose, O-β-D-xylose, O-α-L-ramnose, O-β-D-glucose 6→1-α-L-ramnose.

15. Compounds according to claim 1 that are derivatives of canferol according the general formula (IIB) 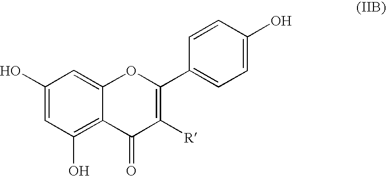
in which R′ is selected in the group of compounds having the following substitutes:
R′═OH, O-β-D-glucose, O-β-D-galactose, O-β-D-xylose, O-α-L-ramnose, O-β-D-glucose 6→1-α-L-ramnose.

16. Compounds according to claim 1 that are derivatives of luteolin according the general formula (IIC) 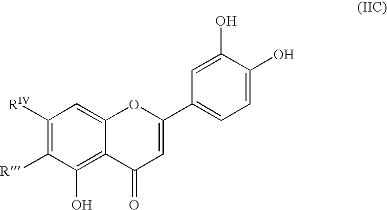
in which:
R′″═H, OH, C-β-D-glucose, C-β-D-glucose-2→1-O-α-L-ramnose R^{IV}═OH, O-β-D-glucose.

17. Compounds according to claim 1 that are derivatives of apigenin according to the general formula (II D)

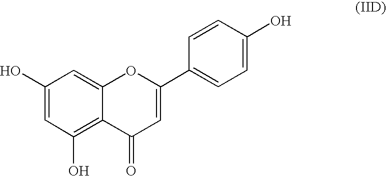

18. Compounds according to claim 1 that are derivatives of quercetagetin according to the general formula (IIE)

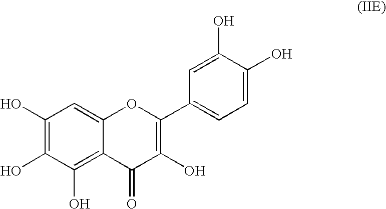

19. Compounds according to claim 1 that are derivatives of fisetin according to the general formula (IIF) 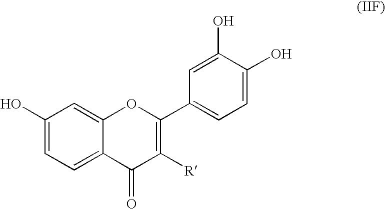
in which R′ is selected in the group of compounds having the following substitutes:
R′═OH, O-β-D-glucose, O-β-D-galactose, O-β-D-xylose, O-α-L-ramnose.

20. Compounds according to claim 1 according to the general formula (IIG) 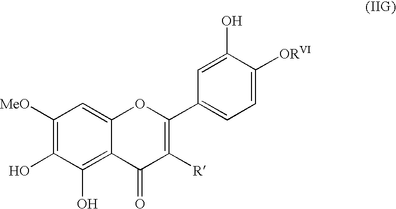
in which:
R′═OH, R^{VI}═H;
R′═O-β-D-glucose, R^{VI}═H;
R′═OH, R^{VI}=β-D-glucose;
R′═O-(β-D-glucose1→4-O-(β-D-glucose), R^{VI}═H;
R′═O-(α-L-ramnose1→2-O-(β-D-glucose), R^{VI}═H;
R′═O-[(2-caffeoil)-β-D-glucose1→4-O-(β-D-glucuronic], R^{VI}═H.

21. Compounds according to claims 1-20 in their diastereoisomeric forms, pure and/or pure enantiomer.

22. Compounds according to claims 1-21 to be used for dermatological use.

23. Compounds according to claims 1-21 to be used for cosmetic use.

24. Compounds according to claims 1-21 to be used to modify MPS increasing the synthesis of stress proteins.

25. Flavonoidic compounds obtained by extraction and purification from plant material to be used for dermatological or cosmetic use to treat ailment and non-esthetic modifications correlated to a change in MPS of euchariotic celles.

26. Products according to claim 25 in which the cells belong to higher animals, including mammals and human.

27. Products according to claims 25 and 26 in which the plant material is chosen among the following plants: Anadenanthera macrocarpa, Potentilla viscosa, Calliandra haematocephala, Guibourtia coleosperma, Eriocaulaceae, Paepalanthus latipes, Paepalanthus velloizioides, Camelia sinensis, and related mixtures.

28. Products according to claims 25 and 26 chosen in the following group: (+)-catechin (2R,3S), (−)-epicatechin (2R,3R), (−)-catechin (2S,3R), (+)-epicatechin (2S,3S), efzelechin (2R,3S), fisetinidol (2R,3S), guibourtinidol (2R,3S), and corresponding glycosids, (−)-catechin-3-gallate (2S,3R), (−)-epicatechin-3-gallate (2R,3R), quercetagetin-7-metil ether, quercetagetin-7-methyl ether-3-O [2-O-caffeoil-β-D-glucopyranosil (1→4)-O-β-D glucuronopyranoside], quercetagetin-7-methyl etere-3-O-neohesperidoside, quercetagetin-7-methyl etere-4′-O-β-D-glucopyranoside, quercetagetin-7-methyl ether-3-O-β-D-glucopyranoside, 6-idroxyluteblin-7-O-β-D-glucopiranoside, luteolin-6-C-β-D-glucopyranosid, fisetinidol-3-O-β-D-xylopiranoside, luteolin-6-C-[α-L-ramnopyranosil-(1→2)-O]-β-D-glucopyranoside, and corresponding mixtures.

29. Pharmaceutical compositions comprising as active principle an effective amount of a compound according to claims 1-28, pharmaceutically acceptable corresponding to derivatives and/or salts, comprising the corresponding optically active compounds in their enantiomers and/or pure diastereoisomers and corresponding mixtures, to prevent or treat illness connected to a change in MPS of eukaryotic cells.

30. Compositions according to claims 29 in which the cells belong to higher animals, including mammals and humans.

31. Compositions according to claims 28 and 29 in which the amount of the active principle ranges between 0.1 and 99.5% in weight.

32. Compositions according to claims 28-31 further comprising: eccipients, diluents, stabilizers, or other adjuvants such to obtain composition to be administered orally, parenterally, rectally, topically, spray.

33. Compositions according to claims 28-32 as pills, tablets, granules, syrup, solution, suspension, creams, ointments, gels, powder, delayed and retarded formulation.

34. Compositions according to claim 33 in a cream for topical use.

35. Cosmetic compositions comprising as an active principle an effective amount of a compound according to claims 1-28, corresponding pharmaceutically acceptable derivatives and/or salts, including the corresponding compounds optically active in their forms as enantiomers and/or pure diastereoisomers and relative mixtures, to prevent and/or treat illness connected with changes in MPS.

36. Compositions according to claim 35 in which the cells are those of higher organisms including mammals and humans.

37. Compositions according to claims 35 and 36 in which the amount of the active principle ranges between 0.1 and 99.5% in weight.

38. Compositions according to claims 35-37 further comprising: eccipients, diluents, stabilizers, or other adjuvants.

39. Compositions according to claim 38 for topical use.

40. Process to obtain compounds according to claims 1-11 comprising the following steps: hydrogenation of the starting product calcon (1) (E)-1-(4′-O-metoxymethylfenil)-3-(2″,4″-di-O-metoxymethyl-fenil)-propene in presence of Pd/C to obtain retro-diidrocalcon (2) that by subsequent reduction with NaBH₄, gives 1,3-diarilpropan-1-olo (3) that is converted in (E)-1,3-diarilpropen (4) using SOCl₂ and 1,8-diazabicicle [5.4.0]undec-7-ene (1,8-DBU); from compound (4) by shaking in a diphasic system BuOH: H₂O 1:1 the corresponding sin-diolo (5) is obtained that, by subsequent deprotection and cyclization, yields a un flavanic derivative (6).

41. Process according to claim 40 further comprising the step of acetylation of the flavanic derivatives (6) on the aromatic —OH with subsequent reaction with the halide of the chosen sugar, previously peracetylated, to obtain the corresponding glycoside.

42. Method to modify MPS of eukaryotic cells characterized in that the cells are treated with effective amounts of at least one compound or product chosen among those of claims 1-28 and related mixtures.

43. Method to induce an heat shock response, such ad heat shock, in eukaryotic cells characterized in that the cells are treated with effective amounts of at least one compound or product chosen among those of claims 1-28 and related mixtures.

44. Method to protect eukaryotic cells from stress conditions characterized in that the cells are with effective amounts of at least one compound or product chosen among those of claims 1-28 and related mixtures.

45. Method according to claims 42-44 in which cells are those of higher organisms, including mammals and humans.

46. Method according to claims 42-44 in which cells are those of L929 cell line or human keratinocytes.

47. Method for a cosmetic treatment that uses at least one compound or product chosen according to claims 1-28 and relative mixtures.

48. Use of compounds or products according to claims 1-28 to modify MPS of eukaryotic cells.

49. Use according to claim 48 in which cells are those of higher organisms, including mammals and humans.

50. Use according to claim 48 in which cells are those of L929 cell line or human keratinocytes.

51. Use of compounds and products according to claims 1-28 to induce a heat shock response under stress condition such as, during heat shock, in eukaryotic organisms.

52. Use according to claim 51 in which cells are those of higher organisms, including mammals and humans.

53. Use according to claim 51 in which cells are those of L929 cell line or human keratinocytes.

54. Use of compounds and products according to claims 1-28 to protect eukaryotic organisms from stress.

55. Use according to claim 54 in which cells are those of higher organisms, including mammals and humans.

56. Use according to claim 54 in which cells are those of L929 cell line or human keratinocytes.

57. Use of compounds and products according to claims 1-28 to produce pharmacological agents for the treatment of pathological conditions due to an alteration of MPS of eukaryotic cells.

58. Use according to claim 57 in which cells are those of higher organisms, including mammals and humans.

59. Use according to claim 57 in which cells are those of L929 cell line or human keratinocytes.

60. Use according to claims 57-59 in which the alteration of MPS is due to at least one of the following stress conditions: oxidative stress, localized mechanic stress, osmotic stress, stress due to hypoxia ischemia, heat shock, UV radiations, by toxic compounds and free radicals.

61. Use according to claims 57-60 in which the pathological state is due to an alteration of MPS is in the following group: diabetes, vascular and cardiovascular diseases, coronary and cerebral diseases, allergies, immune and auto immune diseases, of viral or bacterial origin, tumors, skin diseases or of the mucosa, epithelial, renal, trauma, neurodegenerative diseases, dementia, Alzheimer, Parkinson, AIDS, epilepsy, physiological stress, ulcers, dermatitis, psoriasis burns.

62. Use of compounds and products according to claims 1-28 to produce pharmacological agents to treat and/or to prevent one of the following illness:
chronic degenerative illness, cardiac cerebral ischemia, diabetes, vascular and cardiovascular diseases, coronary and cerebral diseases, allergies, immune and auto immune diseases, of viral or bacterial origin, tumors, skin diseases or of the mucosa, epithelial, renal, trauma, neurodegenerative diseases, dementia, Alzheimer, Parkinson, AIDS, epilepsy, physiological stress, ulcers, dermatitis, psoriasis burns.

63. Use of compounds and products according to claims 1-28 to produce cosmetics.

64. Method for a molecular assay to evaluate the activity of chemical compounds that modify MPS for use as pharmaceutical agents, dermatological and/or cosmetic products, such method comprising the following steps:
Preparation of a vector containing a reporter gene coding for luciferase (or GFP, green fluorescent protein) under the control of a stress inducible hsp70 promoter in mammalian or human cells;
genetic transformation of mammalian cell lines with such vectors;
treatment of the cell lines with the chemical compound of interest and subsequent exposure to stress;
assay of the protein product (luciferase or determination of fluorescence of GFP) after exposure to stress;
determination of anisotropy in the same cell lines do determine the changes in MPS.

65. Method according to claim 64 in which, rather than using a reporter gene heat shock gene transcription is determined by Northern blot directly measuring hsp70 mRNA.

66. Method according to claim 64 in which, rather than using a reporter gene, heat shock gene transcription is determined by quantitative determination of hsp70 mRNA.

67. Method according to claim 64 in which the cell lines are fibroblasts and keratinocytes.

68. Method according to claim 64 in which the stress is a heat shock for variable periods between 20 min to an hour or more.
